(19) United States
(12) Patent Application Publication
(10) Pub. No.: US 2024/0156344 A1
(43) Pub. Date: May 16, 2024

(54) SKIN-ADHERENT, ULTRA-STRETCHABLE, AND CONFORMAL WEARABLE ELECTROCARDIOGRAPHIC DEVICE AND FABRICATION METHOD

(71) Applicant: The Hong Kong University of Science and Technology, Hong Kong (CN)

(72) Inventors: I-Ming HSING, Hong Kong (CN); Yue LI, Hong Kong (CN)

(21) Appl. No.: 18/548,621

(22) PCT Filed: Jun. 17, 2022
(86) PCT No.: PCT/IB2022/000377

§ 371 Date: Sep. 1, 2023

Related U.S. Application Data
(60) Provisional application No. 63/212,128, filed on Jun. 18, 2021.

(51) Int. Cl. A61B 5/00; A61B 5/257; A61B 5/266; A61B 5/282

(52) U.S. Cl. CPC A61B 5/0006; A61B 5/0022; A61B 5/257; A61B 5/266; A61B 5/282; A61B 2562/125; A61B 2562/164

(57) ABSTRACT

An electrocardiographic (ECG) device and fabrication methods of the ECG device are provided for sensing cardiac activities in a test subject. The ECG device includes a plurality of electrodes and a plurality of bridges connecting adjacent electrodes of the plurality of electrodes. The electrodes each has a structure including a first layer and a third layer disposed above the third layer, a second layer of liquid metal disposed between the first and third layers, and a fourth layer of conductive material disposed on the third layer, and the second layer of liquid metal is electrically connected with the fourth layer of conductive material. Each electrode may include a metal film connecting the second layer of liquid metal with the fourth layer of conductive material. The first and third layers are formed with stretchable material, making the ECG device skin-adherent, highly stretchable, and conformal.

### CROSS-REFERENCE TO RELATED APPLICATION

This application claims the benefit of U.S. Provisional Application Ser. No. 63/212,128, filed Jun. 18, 2021, which is hereby incorporated by reference in its entirety including any tables, figures, or drawings.

### BACKGROUND OF THE INVENTION

Currently, wearable biosensing systems that provide effective routes for monitoring electrophysiological signals are ubiquitous in the fields of medicine and healthcare. For example, ambulatory electrocardiographic devices (Holters) are used when long-term electrocardiography (ECG) monitoring is needed for patients who have episodic heart disease such as paroxysmal atrial arrhythmias, which are difficult to predict and detect during the short recording of standard ECG performed in hospitals [1][2]. In the past decade, many types of wearable devices such as integrated ECG sensors of small size and high flexibility[3][4][5], on-skin E-Tattoos[6][7], smart textile electrodes[8], flexible dry electrodes on adhesive tape[9][10], and wet electrodes based on functionalized conductive polymers[11][12], have been developed for long-term cardiac health monitoring.

However, these single-lead ECG sensors or multi-lead electrodes with fixed positions often fail to provide accurate ECG waveforms for disease diagnosis, due to lack of flexible electrodes placement according specific lead method, which is required for providing sufficient vector information for accurate diagnosis of specific cardiac disease symptoms[13][14]. Particularly, these ECG devices can rarely achieve performance of a standard 12-lead ECG method, which typically involves 10 electrodes on the particular position of patient's body that simultaneously measures ECG in 12 different vectors. According to the lead method for diagnosis, 6 electrodes (V1-V6) are placed on exact positions on rib cage and remaining 4 electrodes are respectively placed on right arm (RA), left arm (LA), right leg (RL) and left leg (LL). The 12-lead ECG is universally accepted as a “gold standard” for arrhythmia diagnosis and also plays an important role for myocardial ischemia detection. Since both diseases may appear intermittently, it is essential to develop a wearable multi-lead ECG electrode patch device that is transformable for electrode position adjustment, long-term adhesive for robust signal sensing, and soft and conformal for patient's comfort. Unfortunately, no conventional wearable multi-lead ECG patch devices satisfactorily meet these demands.

### BRIEF SUMMARY OF THE INVENTION

There continues to be a need in the art for improved designs and techniques for a wearable multi-lead electrode patch device and methods for fabricating the patch device for acquiring and monitoring electrical activities of hearts.

Embodiments of the subject invention pertain to a system and methods for an electrocardiographic (ECG) device for sensing cardiac activities in a test subject. The electrocardiographic device comprises a plurality of electrodes; and a plurality of bridges connecting adjacent electrodes of the plurality of electrodes; each electrode of the plurality of electrodes has a structure comprising a first layer and a third layer disposed above the first layer, a second layer of liquid metal disposed between the first and third layers, and a fourth layer of conductive material disposed on the third layer, and the second layer of liquid metal is electrically connected with the fourth layer of conductive material. Moreover, each electrode may further comprise a metal film, the metal film connects the second layer of liquid metal with the fourth layer of conductive material, and the metal film is made of copper. The first and third layers are formed with stretchable material such that the electrocardiographic (ECG) device is skin-adherent, highly stretchable, and conformal. Each of the stretchable first and third layers is formed with Ecoflex material. Each of the first layers of the electrodes is formed with at least one microchannel on a top surface of the first layer for containing the liquid metal of the second layer. Each of the plurality of bridges has a structure comprising a stretchable first layer, a stretchable third layer disposed above the third layer, and a second layer of liquid metal disposed between the first and third layers, wherein the stretchable first layer has at least one microchannel formed on a top surface of the stretchable first layer and the layer of liquid metal is disposed in the at least one microchannel. The conductive material of the fourth layer is conductive hydrogel. Further, the stretchable first layer, the second layer of liquid metal, and the stretchable third layer of each bridge of the plurality of bridges is connected with the first layer, the second layer of liquid metal, and the third layer of the electrodes that the bridge connects, respectively. The second layers of liquid metal of the electrodes are connected with external data acquisition devices for receiving and/or transmitting electric signals. Each of the third layers of the electrodes is formed with a recess for containing the fourth layer. The plurality of electrodes may comprise ten electrodes.

According to an embodiment of the subject invention, a method for fabricating an electrocardiographic (ECG) device is provided. The method comprises degassing a stretchable material, applying the stretchable material into molds, and heating the molds having the stretchable material at a predetermined temperature for solidification; curing and peeling off a first layer from the molds and forming at least one microchannel on the first layer; adding a liquid metal material into the at least one microchannel; forming a second layer of the degassed stretchable material; placing the second layer on top of the first layer containing the liquid metal material for bonding; inserting a metal film through a through-hole of the second layer to contact the liquid metal material; treating the second layer with a benzophenone (BP) solution to obtain a solid interface; washing and drying the structure obtained from above steps; applying the BP solution onto surfaces of the structure for a predetermined period at room temperature; and washing and drying the surfaces of the structure; applying a hydrogel solution onto a top surface of the BP-treated structure; and immediately treating the structure by UV irradiation from bottom. The stretchable material may be Ecoflex 00-30. The liquid metal material may be EGaIn. The metal film may be made of copper. The BP solution may be formed by dissolving BP in a solvent which is formed by 65% acetone and 35% DI water. The BP solution has a concentration of BP of 10 wt %. Moreover, the hydrogel solution is prepared by dissolving an amount of dopamine (DA) powder in DI water and then adding an amount of NaOH aqueous solution; keeping stirring the mixture obtained in room environmental conditions for a predetermined period to allow DA self-polymerize to polydopamine (PDA) chains via an alkali-induced prepolymerization process; adding acrylamide monomer, ammonium persulfate, and N,N′-Methylenebisacrylamide solution into the PDA solution under stirring in ice bath for a predetermined period for uniform dispersing; and mixing an amount of glycerol with DI water to form a glycerol-water binary solvent and adding the mixture into the PDA solution and then adding tetramethylethylenediamine.

In certain embodiments of the subject invention, an electrocardiographic system is provided, comprising an electrocardiographic (ECG) device described above for sensing cardiac activities in a test subject; one or more electronic components receiving ECG signals from the electrodes of the electrocardiographic (ECG) device; and a processing component receiving electric signals transmitted from the one or more electronic components and processing the received electric signals.

### BRIEF DESCRIPTION OF THE DRAWINGS

FIG. 1 is a schematic representation of a skin-adherent, ultra-stretchable, and conformal wearable electrocardiographic device for ambulatory monitoring, the wearable electrocardiographic device having an arrangement of a 12-lead electrocardiography (ECG) patch including a plurality of electrodes, according to an embodiment of the subject invention.

FIG. 2 is an explosive view of the electrodes of the wearable electrocardiographic device, according to an embodiment of the subject invention.

FIG. 3 is a schematic representation of the interconnects of the wearable electrocardiographic device, according to an embodiment of the subject invention.

FIG. 4A and FIG. 4B are schematic representations of the wearable electrocardiographic device, showing the size parameters of the 12-lead ECG patch, according to an embodiment of the subject invention.

FIG. 5 is a schematic representation of a method for fabricating the wearable electrocardiographic device, according to an embodiment of the subject invention.

FIG. 6 is a schematic representation illustrating the wearable electrocardiographic device of FIG. 1 attached to a test subject and coupled to a wireless ECG system for ambulatory monitoring, according to an embodiment of the subject invention.

FIG. 7A shows a 3 single-lead electrode electrocardiographic device having the stretched/unstretched channels and FIG. 7B shows a data plot of the ECG recordings obtained by the 3 single-lead electrodes electrocardiographic device, according to an embodiment of the subject invention.

### DETAILED DISCLOSURE OF THE INVENTION

The embodiments of subject invention show a wearable multi-lead electrocardiographic (ECG) device that is skin-adherent, high-stretchable, and conformal and methods for fabricating the ECG device.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

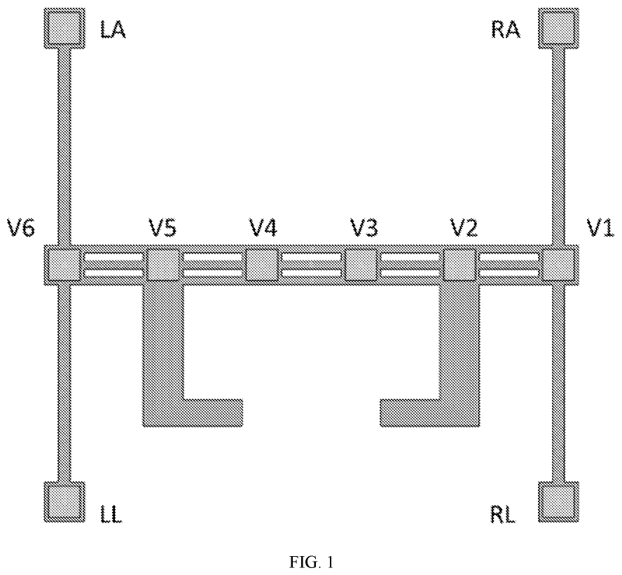

Referring to FIG. 1, a wearable 12-lead ECG electrocardiographic device having 10 electrodes including V1, V2, V3, V4, V5, V6, LA, RA, RL, and LL, is shown. The electrocardiographic device is configured to be stretchable, allowing a position of the individual electrode to be adjustable. The electrodes of the electrocardiographic device are connected by a plurality of bridges having liquid metal embedded within. Each of the plurality of bridges comprises at least one microchannel formed on an elastomer substrate of the bridge. The liquid metal is injected into the at least one microchannel of the bridge. Accordingly, the electrocardiographic device can be readily adhered onto the chest of a test subject for ECG testing in real-world applications.

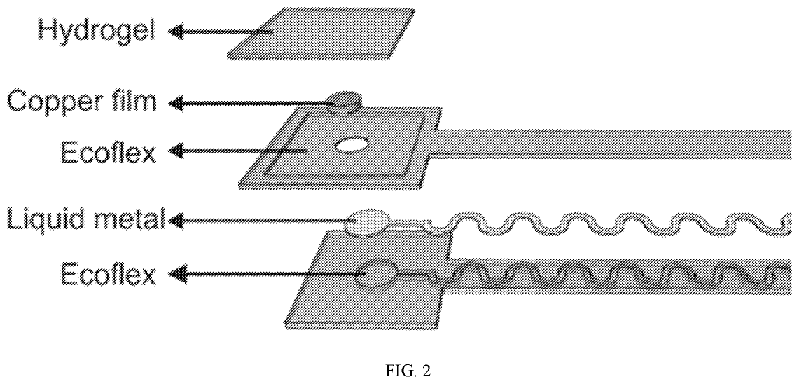

As illustrated in FIG. 2, the electrocardiographic device includes four layers. The liquid metal interconnect is sandwiched between two Ecoflex layers and connected with a layer of conductive hydrogel by a metal thin film.

In one embodiment, the metal thin film is a copper thin film. In certain embodiments, the metal thin film can be made by one of platinum, gold, silver, silver chloride, and conductive stainless steel. In one embodiment, the liquid metal interconnect is made of one of liquid metals including gallium, gallium alloys, and mercury.

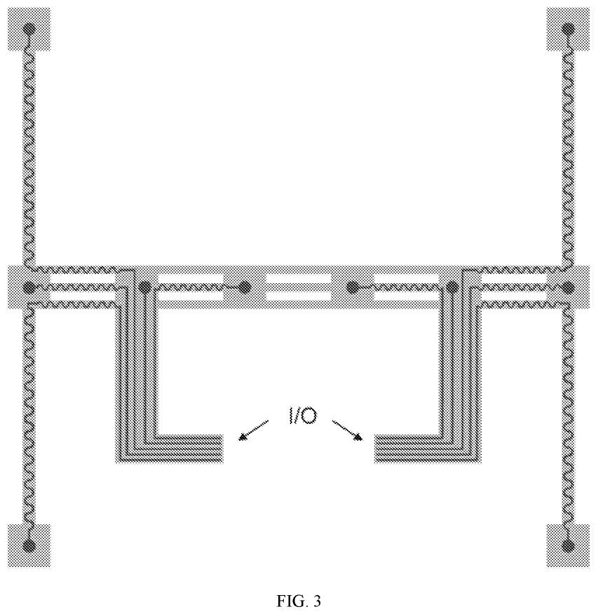

Referring to FIG. 3, the liquid metal interconnect is embedded in the bridge and connected with external data acquisition device of the ECG system via I/O ports. The electrodes V4, V5, LA, V6, LL can be disposed on one side (from top to bottom) of the ECG device and the electrodes V3, V2, RA, V1, RL can be disposed on the opposite side (from top to bottom) of the ECG device.

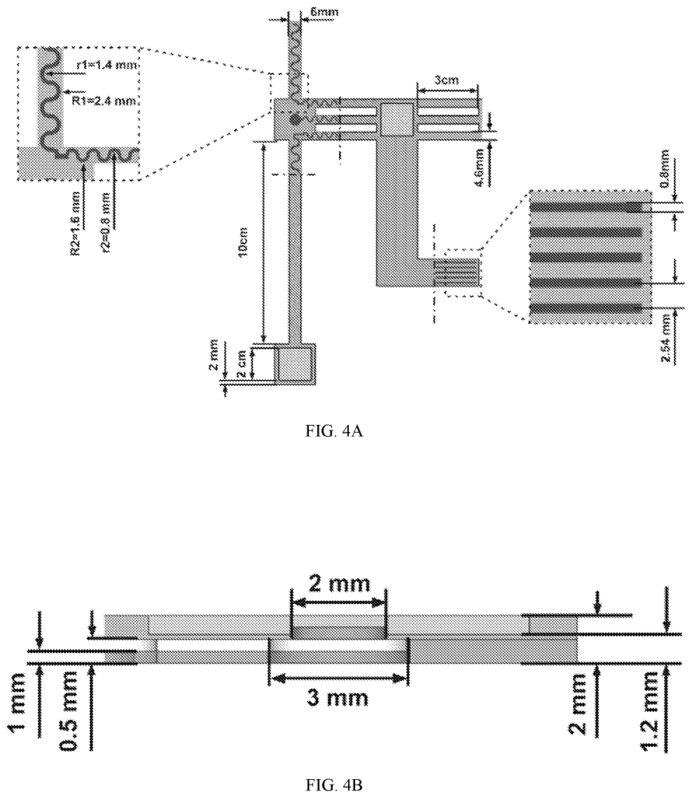

In one embodiment, as illustrated in FIG. 4A, four bridges are connected to LA, LL, RA, and RL and each bridge has a dimension of 10 cm in length and 6 mm in width. The width of each interconnect is 1 mm, the inside and outside diameters of serpentine microchannels are r1=1.4 mm and R=2.4 mm, respectively. For the 3-bridge structure connected V1 to V6, each of the bridges is formed with a width of 4.6 mm and a length of 3 cm. The interconnects in the 3-bridge structure each has a dimension of 0.8 mm in width and the inside and outside radii of serpentine microchannels are r1=0.8 mm and R=1.6 mm, respectively. The I/O pads are designed to connect with standard 2.54 mm pin header, with a distance of 2.54 mm between the center of each microchannels.

It is noted that the patch is highly stretchable and can compliantly adjust according to the motions of the test subject. However, the deformation of the conductive path, especially the cross-section area of the interconnects, may change the resistance of the wires and thus affect the detected signals. Therefore, the interconnects may be formed with a serpentine shape. The curves of the serpentine shape change the shape against the stretching, instead of changing the cross-section of the path such that a stable electrical property is maintained during deformation of the patch.

FIG. 4B shows a cross-sectional view of the electrode of the ECG device. The total thickness of the electrode is 2 mm, including a thickness of 1.2 mm for the substrate which may be made of Ecoflex and a thickness of 0.8 mm for the hydrogel layer. The substrate has two layers including a first layer with at least one microchannel at bottom and a second layer with a recess in a shape of, for example, a square, for containing the hydrogel. Each of the two layers has a thickness of 1 mm. The depth of microchannel for containing liquid metal is 0.5 mm and the diameter of the liquid metal cylinder at center of electrode is 3 mm. A copper thin film with a diameter of 2 mm is inserted through a through-hole of the Ecoflex into the liquid metal for connecting the liquid metal with the hydrogel. In one embodiment, the substrate may be made of flexible and stretchable elastomer polymers such as rubber, parylene-C, polydimethyl-siloxane Sylgard 184, polyurethane, latex, or VHB.

In one embodiment, the hydrogel layer may be made of conductive hydrogels, such as PAM/PEGDA/PVA/PAA based hydrogels.

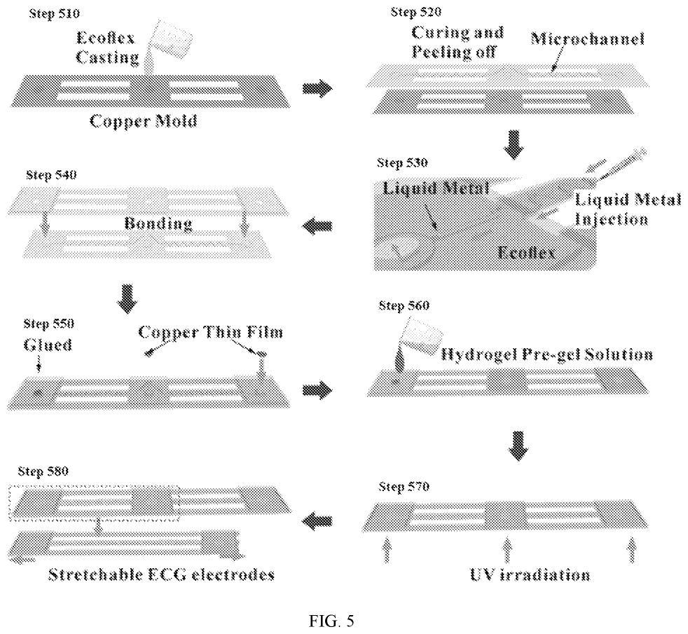

FIG. 5 shows a method for fabricating the wearable electrocardiographic device in which two customized copper molds are made to pattern the two layers of Ecoflex film that includes a first layer formed with at least one microchannel and a second layer formed with a recess for containing the hydrogel.

The method comprises seven steps. First, at step 510, material Ecoflex 00-30 is degassed and poured into the molds followed by heating at 60° C. in an oven. After complete solidification, at step 520, a first layer is peeled off from the molds and pressed by a glass slide previously cleaned with isopropanol to form a microchannel. Excessive pressure should be avoided in this step to prevent causing damages to the microchannel.

Then, at step 530, material EGaIn is injected into the microchannel with a syringe, and the glass slide is removed thereafter. A thin layer of degassed Ecoflex is then poured on top of the first layer containing the microchannel metal filled with the liquid metal, followed by placing the second layer on the top the first layer for bonding at step 540.

Next, at step 550, a thin copper film is inserted through the second layer to contact the liquid metal and sealed with Kafuter glue at the center of electrode parts of the Ecoflex substrate. The Ecoflex substrate is then treated by a benzophenone (BP) solution to achieve a robust interface with the hydrogel.

The electrode structure is next thoroughly washed with methanol and deionized (DI) water and blow-dried with N₂ gas. Then, a BP solution is applied onto the Ecoflex surface covering the entire electrode structure for 2 minutes at room temperature. The BP solution may be formed by dissolving BP in a solvent which is formed by 65% acetone and 35% DI water. The BP solution has a concentration of BP of 10 wt %. After the BP treatment, the Ecoflex surface is washed with methanol and completely blow-dried by N₂ gas.

Then, at step 560, the hydrogel is prepared as described below.

0.02 g of dopamine (DA) powder is first dissolved in 5 ml of DI water prior to addition of 300 μl of 1.5M NaOH aqueous solution. Stirring of the mixture is kept in room environmental conditions for 20 minutes to let the DA self-polymerize to PDA chains via an alkali-induced prepolymerization process. Next, 2.5 g of acrylamide monomer, 0.25 g of ammonium persulfate, and 200 μl of N,N′-Methylenebisacrylamide solution (2% in H₂O) are added into the PDA solution under stirring in ice bath for 10 minutes, allowing the components to uniformly disperse. Then, 2 ml of glycerol are mixed with 2 ml of DI water to form a glycerol-water binary solvent and added into the PDA solution, followed by 20 μl of tetramethylethylenediamine.

Next, at step 570, after stirring for a few seconds, the solution is poured into the BP-treated electrode structure on the Ecoflex substrate and immediately treated by UV irradiation from the bottom due to the UV-shielding property of PDA. During the UV treatment, the monomer polymerizes to form the hydrogel and a robust interfacial hybrid is obtained between the hydrogel and the elastomer. As a result, at step 580, a wearable ECG electrocardiographic device that is skin-adherent, highly stretchable, and conformal is obtained.

The wearable ECG device includes silicon rubber elastomer as water-proof cover, liquid material as electrical interconnect, and ultra-stretchable polydopamine-based hydrogel as adhesive layer. Moreover, the ECG patch comprises at least two electrodes and connected with bridges to provide inhomogeneous deformation for electrical stability and skin-electrode robustness during stretching.

### Materials and Methods

In the two examples below, embodiments of the wearable ECG electrocardiographic devices are employed for detecting real-time ECG signals to perform remote diagnosis.

### Example 1: Wearable ECG Electrocardiographic Device for Remote Diagnosis

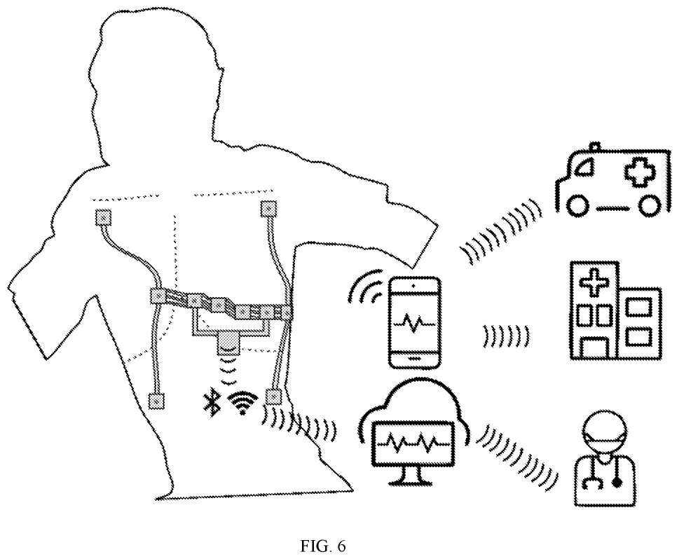

FIG. 6 is a schematic representation of one exemplary embodiment of the 12-lead ECG electrocardiographic device of the subject invention working with a wireless monitoring system. A running test subject has the ECG electrocardiographic device adhered upon his/her chest to monitor the ECG signals. The electrodes of the ECG electrocardiographic device can be stretched and adhered on the exact positions. In addition, the ECG electrocardiographic device may connect to one or more electronic components that receive ECG signals from the electrodes of the ECG device and transmit the ECG signals to a receiving unit or a remote communications station or any suitable device. The corresponding signals are transferred preferably via radio wave. The receiving unit can be an ECG monitor, a cell phone, a computing device, or any other type of device that relays and/or processes the received signals and can optionally transmit instructions to another remote location such as a doctor's office or any other monitoring service.

### Example 2: Single-Lead Electrode Electrocardiographic Device for Real-Time ECG Monitoring

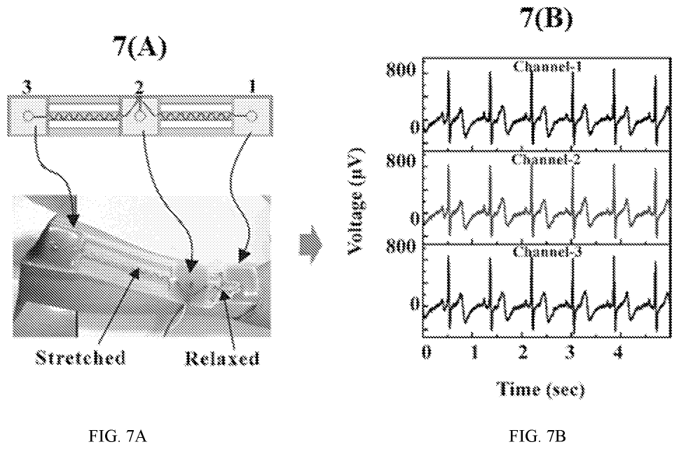

In another embodiment of the subject invention, the method for fabricating the ECG electrocardiographic device described above can be utilized to fabricate ECG electrocardiographic device with different leads by simply changing the copper molds for elastomer and the corresponding sensor systems. Thus, a 3 single-lead electrodes ECG electrocardiographic device can be fabricated for measuring real-time ECG signals as shown in FIGS. 7A and 7B.

The embodiments of the invention are directed towards a highly stretchable ECG electrocardiographic device with self-adhesive hydrogel-based electrodes, achieving a superior compliance along the skin in daily routine. To realize the stretchable ECG electrocardiographic device with stable ECG signal measurements, the ECG electrocardiographic device is designed with a 3-bridge structure to allow minimal deformation of the electrode part, while the main deformation occurs preferably on the bridges. Liquid metal, such as eutectic gallium-indium (EGaIn), which has excellent electrical stability under large stretching deformation, is injected into the microchannels of the bridges to serve as interconnects. The 3-bridge structure which provides inhomogeneous stress and deformation distribution can also reduce the shear force of the hydrogel adhesive layer for robust skin-electrode adhesion. Moreover, a glycerol—water mixture is used as the binary solvent for PDA-PAM hydrogel to keep a long-term mechanical stability in different conditions. The ECG electrocardiographic device with stretchability, flexibility and adhesiveness provides a robust, conformal and comfortable skin-electrode interface for collecting stable ECG signals when the test subjects are moving or sweating. Further, the scalable design and adjustable position of the electrodes allow the development of a 15-lead or other multi-lead ECG Holters for the ECG electrocardiographic device. The stretchable interconnect is embedded in the ECG electrocardiographic device to minimize noise generated by the shaking of lead wires that are connected out to a signal processing system, enabling ECG signal monitoring during a sport event.

The embodiments of the subject invention provide following key advantages.
Key Advantage 1: the design of interconnect of bridge of the stretchable ECG electrocardiographic device provides inhomogeneous strain distribution under stretching.
Key Feature 2: the interconnects of bridges are fabricated by filling liquid metal into serpentine microchannels for stretchability and electrical stability.
Key Feature 3: the self-adhesive layer is made of a polydopamine/polyacrylamide glycerol-water hydrogel for long-term usability.

Therefore, the wearable multi-lead ECG electrocardiographic device is skin-adherent, high-stretchable, and conformal. The superior properties of the ECG electrocardiographic device are achieved, in part, due to the inhomogeneous deformation of the “3-bridge” structure containing liquid metal interconnects that simultaneously maintain high conductivity and stable resistance during cycling stretching. These features ensure a stable ECG signal measurement when the ECG electrocardiographic device is subjected to deformations during daily use or placed on different parts of the body according to different lead methods. Moreover, the ECG electrocardiographic device is scalable, as the remaining bridges without interconnects on the 3-bridge structure are reserved for connecting more electrodes to the ECG electrocardiographic device. The device can be covered by biocompatible and stretchable silicon rubber Ecoflex which serves as a passivation layer and achieves waterproof effects. To realize a conformal and robust skin-electrode interface, the adhesive layer is formed by a polydopamine (PDA)-based glycerol-hydrogel with high stretchability and good tissue adhesiveness in various measurement conditions. The interconnects of the ECG electrocardiographic device can be connected to an external wireless data acquisition system to minimize the influence of the lead wires. Accordingly, the ECG electrocardiographic device provides a comfortable and robust skin-electrode interface with great signal stability during long-term ECG measurements for healthcare monitoring or medical practice.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] S. Chen, J. Qi, S. Fan, Z. Qiao, J. C. Yeo, Adv. Healthc. Mater. 2021, 2100116, 1-23.
[2] J. P. DiMarco, J. T. Philbrick, Ann. Intern. Med. 1990, 113, 53-68.
[3] S. P. Lee, G. Ha, D. E. Wright, Y. Ma, E. Sen-Gupta, N. R. Haubrich, P. C. Branche, W. Li, G. L. Huppert, M. Johnson, H. B. Mutlu, K. Li, N. Sheth, J. A. Wright, Y. Huang, M. Mansour, J. A. Rogers, R. Ghaffari, npj Digit. Med. 2018, 1, 1-8.
[4] E. Fung, M.-R. JÃ [symbol] rvelin, R. N. Doshi, J. S. Shinbane, S. K. Carlson, L. P. Grazette, P. M. Chang, R. S. Sangha, H. V. Huikuri, N. S. Peters, Front. Physiol. 2015, 6, 149.
[5] Y. Chuo, M. Marzencki, B. Hung, C. Jaggernauth, K. Tavakolian, P. Lin, B. Kaminska, IEEE Trans. Biomed. Circuits Syst. 2010, 4,281-294.
[6] J. Alberto, C. Leal, C. Fernandes, P. A. Lopes, H. Paisana, A. T. de Almeida, M. Tavakoli, Sci. Rep. 2020, 10, 1-11.
[7] W. H. Yeo, Y. S. Kim, J. Lee, A. Ameen, L. Shi, M. Li, S. Wang, R. Ma, S. H. Jin, Z. Kang, Y. Huang, J. A. Rogers, Adv. Mater. 2013, 25, 2773-2778.
[8] K. Arquilla, A. Webb, A. Anderson, Sensors. 2020, 20, 1013.
[9] C. Lou, R. Li, Z. Li, T. Liang, Z. Wei, M. Run, X. Yan, X. Liu, Sensors. 2016, 16, 1833.
[10] D. Lai, Y. Bu, Y. Su, X. Zhang, C. S. Ma, IEEE Sens. J. 2020, 20, 12295-12306.
[11] T. Shay, O. D. Velev, M. D. Dickey, Soft Matter. 2018, 14, 3296-3303.
[12] J. Li, L. Geng, G. Wang, H. Chu, H. Wei, Chem. Mater. 2017, 29, 8932-8952.
[13] Y. Li, Y. Luo, S. Nayak, Z. Liu, O. Chichvarina, E. Zamburg, X. Zhang, Y. Liu, C. H. Heng, A. V. Y. Thean, Adv. Electron. Mater. 2019, 5, 1800463.
[14] H. Zhang, L. Tian, H. Lu, M. Zhou, H. Zou, P. Fang, F. Yao, G. Li, in Proc. Annu. Int. Conf. IEEE Eng. Med. Biol. Soc. EMBS, Institute Of Electrical And Electronics Engineers Inc. 2017.

1. An electrocardiographic (ECG) device for sensing cardiac activities in a test subject, the electrocardiographic device comprising:
a plurality of electrodes; and
a plurality of bridges connecting adjacent electrodes of the plurality of electrodes;
wherein each electrode of the plurality of electrodes has a structure comprising a first layer and a third layer disposed above the first layer, a second layer of liquid metal disposed between the first and third layers, and a fourth layer of conductive material disposed on the third layer, and
wherein the second layer of liquid metal is electrically connected with the fourth layer of conductive material.

2. The electrocardiographic (ECG) device of claim 1, each electrode further comprising a metal film.

3. The electrocardiographic (ECG) device of claim 2, wherein the metal film connects the second layer of liquid metal with the fourth layer of conductive material.

4. The electrocardiographic (ECG) device of claim 2, wherein the metal film is made of copper.

5. The electrocardiographic (ECG) device of claim 1, wherein the first and third layers are formed with stretchable material such that the electrocardiographic (ECG) device is skin-adherent, stretchable, and conformal.

6. The electrocardiographic (ECG) device of claim 5, wherein each of the stretchable first and third layers is formed with Ecoflex material.

7. The electrocardiographic (ECG) device of claim 1, wherein each of the first layers of the electrodes is formed with at least one microchannel on a top surface of the first layer for containing the liquid metal of the second layer.

8. The electrocardiographic (ECG) device of claim 1, wherein each of the plurality of bridges has a structure comprising a stretchable first layer, a stretchable third layer disposed above the first layer, and a second layer of liquid metal disposed between the first and third layers, wherein the stretchable first layer has at least one microchannel formed on a top surface of the stretchable first layer and the layer of liquid metal is disposed in the at least one microchannel.

9. The electrocardiographic (ECG) device of claim 1, wherein the conductive material of the fourth layer is conductive hydrogel.

10. The electrocardiographic (ECG) device of claim 7, wherein the stretchable first layer, the second layer of liquid metal, and the stretchable third layer of each bridge of the plurality of bridges is connected with the first layer, the second layer of liquid metal, and the third layer of the electrodes that the bridge connects, respectively.

11. The electrocardiographic (ECG) device of claim 1, wherein the second layers of liquid metal of the electrodes are connected with external data acquisition devices for receiving and/or transmitting electric signals.

12. The electrocardiographic (ECG) device of claim 1, wherein each of the third layers of the electrodes is formed with a recess for containing the fourth layer.

13. The method of claim 1, wherein the plurality of electrodes comprise ten electrodes.

14. A method for fabricating an electrocardiographic (ECG) device, the method comprising:
degassing a stretchable material, applying the stretchable material into molds, and heating the molds having the stretchable material at a predetermined temperature for solidification;
curing and peeling off a first layer from the molds and forming at least one microchannel on the first layer;
adding a liquid metal material into the at least one microchannel;
forming a second layer of the degassed stretchable material;
placing the second layer on top of the first layer containing the liquid metal material for bonding;
inserting a metal film through a through-hole of the second layer to contact the liquid metal material;
treating the second layer with a benzophenone (BP) solution to obtain a solid interface;
washing and drying a structure obtained from the foregoing steps;
applying the BP solution onto surfaces of the structure for a predetermined period at room temperature to yield a BP-treated structure;
washing and drying the surfaces of the BP-treated structure;
applying a hydrogel solution onto a top surface of the BP-treated structure to yield a resulting structure; and
immediately treating the resulting structure by UV irradiation from bottom.

15. The method of claim 14, wherein the stretchable material is Ecoflex 00-30.

16. The method of claim 14, wherein the liquid metal material is EGaIn.

17. The method of claim 14, wherein the metal film is made of copper.

18. The method of claim 14, wherein the BP solution is formed with 10 wt. % of benzophenone.

19. The method of claim 14, wherein the hydrogel solution is prepared by:
dissolving an amount of dopamine (DA) powder in DI water and then adding an amount of NaOH aqueous solution;
keeping stirring the mixture obtained in room environmental conditions for a predetermined period to allow DA self-polymerize to polydopamine (PDA) chains via an alkali-induced prepolymerization process;
adding acrylamide monomer, ammonium persulfate, and N,N′-Methylenebisacrylamide solution into the PDA solution under stirring in an ice bath for a predetermined period for uniform dispersing; and
mixing an amount of glycerol with DI water to form a glycerol-water binary solvent and adding the mixture into the PDA solution and then adding tetramethylethylenediamine.

20. An electrocardiographic system comprising:
an electrocardiographic (ECG) device of claim 1 configured to sense cardiac activities in a test subject;
one or more electronic components configure to receive ECG signals from the electrodes of the electrocardiographic (ECG) device; and
a processing component configured to receive electric signals transmitted from the one or more electronic components and to process the received electric signals.
